(19) United States
(12) Patent Application Publication
(10) Pub. No.: US 2021/0282952 A1
(43) Pub. Date: Sep. 16, 2021

(54) ENDOVASCULAR CATHETER WITH DELIVERY SYSTEM SEPARATELY ASSEMBLED TO STENT GRAFT SYSTEM

(71) Applicant: Medtronic Vascular, Inc., Santa Rosa, CA (US)

(72) Inventor: John D. WELTER, Santa Rosa, CA (US)

(21) Appl. No.: 16/818,644

(22) Filed: Mar. 13, 2020

(51) Int. Cl. A61F 2/962; A61F 2/07

(52) U.S. Cl. CPC A61F 2/962; A61F 2002/826; A61F 2/07

(57) ABSTRACT

An endovascular catheter system includes a stent graft system and a delivery system that are separately connected to one another. In embodiments, the stent graft system includes a stent graft configured to expand radially outwardly, and a stent graft cover surrounding at least a portion of the stent graft and configured to maintain the stent graft in a constricted configuration. The stent graft cover can slide relative to the stent graft to enable the stent graft to expand radially outward. A hollow stent graft middle member is located radially inward of the stent graft cover. The delivery system is coupled to the stent graft system and includes a delivery system outer cover configured to assemble to the stent graft cover.

### TECHNICAL FIELD

The present disclosure relates to medical implantation devices, particularly, a stent graft delivery system and method of use, more specifically an endovascular catheter with a separately-assembled delivery system and stent graft system.

### BACKGROUND

The use of endovascular procedures has been established as a minimally invasive technique to deliver a variety of clinical treatments in a patient's vasculature. A stent graft is an implantable device made of a tube-shaped surgical graft covering and an expanding or self-expanding frame. The stent graft is placed inside a blood vessel to bridge, for example, an aneurismal, dissected, or other diseased segment of the blood vessel, and, thereby, exclude the hemodynamic pressures of blood flow from the diseased segment of the blood vessel.

### SUMMARY

In one embodiment, an endovascular catheter includes a stent graft system and a delivery system. The stent graft system includes a stent graft configured to expand radially outwardly; a stent graft cover surrounding at least a portion of the stent graft and configured to maintain the stent graft in a constricted configuration, and to slide relative to the stent graft to enable the stent graft to expand radially outward; and a hollow stent graft middle member located radially inward of the stent graft cover. The delivery system is configured to couple to the stent graft system and includes a delivery system outer cover configured to assemble to the stent graft cover via a first connection; and a delivery system middle member configured to assemble to stent graft middle member via a second connection.

In another embodiment, an endovascular catheter assembly includes a stent graft portion having a proximal end, a distal end, an expandable stent graft, and a stent graft cover configured to slide over the stent graft to enable the stent graft to expand radially outward. The catheter assembly also includes a delivery portion having a proximal end, a distal end, a handle at the proximal end of the delivery portion, and an outer sleeve extending from the handle toward the distal end of the delivery portion, wherein the outer sleeve is configured to assemble to the stent graft cover.

In another embodiment, a method of deploying a plurality of stent grafts in one or more vessels of a body includes the following steps: assembling a first stent graft system to a delivery system in which a first stent graft cover is removably coupled to a delivery system outer cover; advancing the first stent graft system to a first deployment site within the body; retracting the first stent graft cover to enable a first stent graft to deploy; withdrawing at least a portion of the first stent graft system and the delivery system away from the body; removing the first stent graft cover from the delivery system outer cover; assembling a second stent graft system to the delivery system in which a second stent graft cover is removably coupled to the delivery system outer cover; advancing the second stent graft system to a second deployment site within the body; and retracting the second stent graft cover to enable a second stent graft to deploy.

### BRIEF DESCRIPTION OF THE DRAWINGS

FIG. 1 is a plan view of an endovascular catheter with a delivery system separated from a stent graft system, according to one embodiment.

FIG. 2 is an exploded assembly view of connections between the delivery system and the stent graft system, according to one embodiment.

FIG. 3 is a cross-sectional view of a snap-fit connection between a portion of the delivery system and a portion of the stent graft system, according to one embodiment.

FIG. 4 is a side view of a snap-fit connection between a portion of the delivery system and a portion of the stent graft system, according to another embodiment.

FIG. 5 is an exploded assembly view of connection between the delivery system and the stent graft system of another embodiment of a catheter.

FIG. 6 is an exploded assembly view of connections between the delivery system and the stent graft system according to another embodiment incorporating a seal.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the embodiments. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Directional terms used herein are made with reference to the views and orientations shown in the exemplary figures. A central axis is shown in the figures and described below. Terms such as “outer” and “inner” are relative to the central axis. For example, an “outer” surface means that the surfaces faces away from the central axis, or is outboard of another “inner” surface. Terms such as “radial,” “diameter,” “circumference,” etc. also are relative to the central axis. The terms “front,” “rear,” “upper” and “lower” designate directions in the drawings to which reference is made.

Unless otherwise indicated, for the delivery system the terms “distal” and “proximal” are used in the following description with respect to a position or direction relative to a treating clinician. “Distal” and “distally” can refer to positions distant from or in a direction away from the clinician, while “proximal” and “proximally” can refer to positions near or in a direction toward the clinician. For the stent-graft prosthesis, or when referring to location within a human anatomy, “proximal” is the portion nearer the heart by way of blood flow path while “distal” is the portion of the stent-graft further from the heart by way of blood flow path.

A stent graft is an implantable device made of a tube-shaped surgical graft covering and an expanding or self-expanding frame. The stent graft is placed inside a blood vessel to bridge, for example, an aneurismal, dissected, or other diseased segment of the blood vessel, and, thereby, exclude the hemodynamic pressures of blood flow from the diseased segment of the blood vessel.

An endovascular catheter (hereinafter referred to as a catheter or delivery catheter) can be used to deliver and deploy a stent graft in the blood vessel. The catheter can include a handle, an elongate outer shaft or stent graft cover, an elongate inner shaft or tubular component, and an optional tip capture device. The stent graft cover and the tip capture device hold a stent graft in a compressed delivery configuration within a distal portion of delivery catheter. To deploy of the stent graft, a sheath retraction mechanism of the handle can be manipulated (e.g. rotated) by the surgical technician to retract the stent graft cover to thereby expose the stent-graft.

The handle, tubes, stent graft, and other components of the catheter are currently shipped and provided to the hospitals and caregivers as a single, integrated mechanism. Various dimensions of catheters and/or stent grafts therefore require an individual stock keeping unit (SKU) for that particular dimensional change. For example, one catheter with a tip having a, e.g., 5 millimeter diameter would have a single SKU for that entire catheter, and another catheter with only a change to a, e.g., 4 millimeter diameter tip would have a separate SKU, even though there is only one minor different between the two devices. Similarly, a stent graft having a length of, e.g., 30 mm would require a whole separate catheter than a stent graft having a length of, e.g., 40 mm. Moreover, shipping of the entire singular catheter can come with high shipping demands.

Therefore, according to various embodiments disclosed herein, an endovascular catheter is provided with separate, attachable sub-systems. For example, a delivery system or delivery portion of the system (including the handle, an outer tube, an inner tube, etc.) can be provided separated from a stent graft system or stent-graft portion of the system (including a stent graft, an outer tube, an inner tube, etc.). The delivery system and the stent graft system can be shipped and provided to the caregivers separately, whereupon they can be assembled by the surgical technician or other operator prior to a surgical procedure. In some embodiments, the outer tube of the delivery system can be assembled to the outer tube of the stent graft system, and the inner tube of the delivery system can be assembled to the inner tube of the stent graft system. The attachment can be removeable, allowing the surgical technician to deploy a first stent graft using the handle, remove the first stent graft assembly from the handle, attach a second stent graft assembly to the handle, and deploy a second stent graft using the same handle. Additional structure, uses, and benefits of the various embodiments shown in the Figures are explained below.

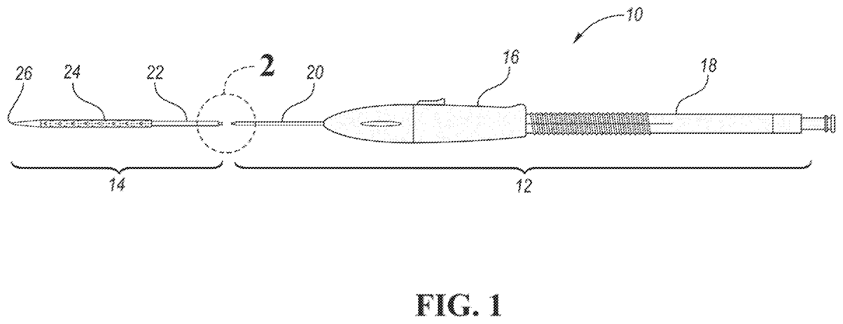

FIG. 1 illustrates a plan view of a catheter 10 for a self-expanding stent-graft prosthesis in accordance with an embodiment hereof. The catheter 10 can be used to treat an aneurysm, such as an abdominal aortic aneurysm (AAA) or a thoracic aortic aneurysm (TAA). The catheter 10 can also be referred to as a delivery catheter or stent-graft delivery system. The catheter 10 includes a delivery subsystem or delivery system 12, and a stent graft subsystem or stent graft system 14. The delivery system 12 includes a handle 16 operatively connected to a stent graft cover retraction mechanism, in which manipulation (e.g., rotation) of the handle relative to a threaded shaft 18 can retract the stent graft cover, allowing the stent graft cover to expand radially outward, as will be described further below. The delivery system 12 also includes one or more lumens 20 that are hollow and configured to enable other tools, lumens, or guide wires to pass therethrough. The stent graft system 14 includes one or more lumens 22 configured to attach to the lumens 20 of the delivery system 12. The stent graft system 14 also includes a stent graft 24 contained within an outer cover, and a tapered tip. As will be explained below, the lumens 20, 22 can include inner and outer shafts configured for attachment or assembly. In one embodiment, a tip capture mechanism (which will be described in one embodiment below) holds a stent graft in a compressed delivery configuration within a distal portion of delivery catheter. During deployment of the stent-graft, the stent graft cover retraction mechanism of the handle can be rotated by the surgical technician in order to proximally retract the stent graft cover to thereby incrementally expose the stent-graft and, once the stent-graft is properly positioned, to permit the full release of the self-expanding stent graft from the catheter.

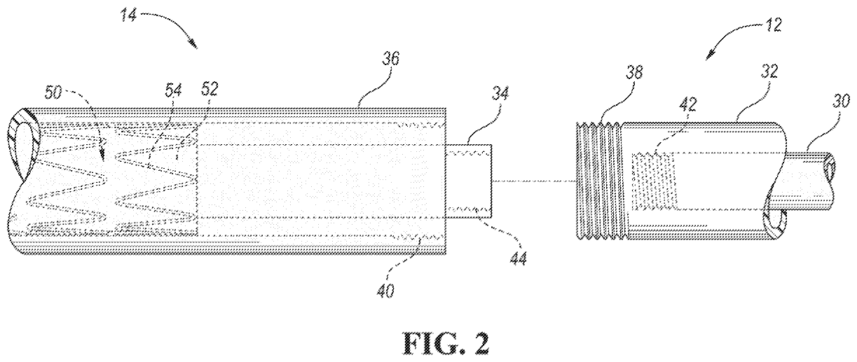

FIG. 2 illustrates an enlarged portion of FIG. 1 at a break between the delivery system 12 and a stent graft system 14 of the catheter 10. The delivery system 12 can also be referred to as a delivery subassembly or delivery portion of the overall catheter assembly, and the stent graft system 14 can also be referred to as a stent graft subassembly or stent graft portion of the overall catheter assembly. The delivery system 12 and the stent graft system 14 can be attached to one another to create the fully-assembled catheter, with the point of attachment shown in FIG. 2. The attachment between the delivery system 12 and the stent graft system 14 can be one of many various types of connections, including a threaded connection (shown in FIG. 2) or a snap-fit connection (described below).

Referring to FIG. 2, the delivery system includes a delivery system middle member 30, and a delivery system outer cover 32 (also referred to as a delivery system sleeve). The delivery system middle member 30 extends along an axis, and may be coaxial with the delivery system outer cover 32. The delivery system outer cover 32 is slideable along the axis relative to the delivery system middle member 30. The delivery system middle member 30 and delivery system outer cover 32 are hollow and configured to enable one or more lumens, tubes or tools to pass therethrough.

Similar to the delivery system 12, the stent graft system 14 has a stent graft middle member 34 and a stent graft cover 36 (also referred to as a stent graft sleeve). The stent graft middle member 34 extends along an axis, and may be coaxial with the stent graft cover. The stent graft cover 36 is slideable along the axis relative to the stent graft middle member 34. The stent graft middle member 34 and stent graft cover 36 are hollow and configured to enable one or more lumens, tubes or tools to pass therethrough. In an embodiment, the stent graft cover 36 is a tubular sheath that may be formed from a composite material having a braided layer of polyether block amide, such as PEBAX®, that is sandwiched between layers of polyamide, such as VESTAMID®. Other materials may be provided in addition or in substitution these. Moreover, the delivery system outer cover 32 may be made of similar material. However, due to the ability of the stent graft system 14 to be removed and attached to the delivery system 12, the delivery system outer cover 32 can be made of a different material than the stent graft cover 36, such as, for example, a material with different (e.g., less) flexibility or stiffness. For example, in one embodiment, the stent graft cover 36 is made of a first material with a first flexibility or stiffness, and the delivery system outer cover 32 is made of a second material with a second flexibility or stiffness that is less flexible or stiff than the first material.

In an embodiment, the delivery system outer cover 32 is attachable to the stent graft cover 36 at a first connection. Likewise, the delivery system middle member 30 is attachable to the stent graft middle member 34 at a second connection. The first and second connections can be threaded connections, for example, shown in FIG. 2. External threads 38 can be machined, molded, or otherwise formed on the outer surface of the delivery system outer cover 32, and internal threads 40 can be similarly formed on the inner surface of the stent graft cover 36. Likewise, external threads 42 can be machined, molded, or otherwise formed on the outer surface of the delivery system middle member 30, and internal threads 44 can be similarly formed on the inner surface of the stent graft middle member 34. While the delivery system components are shown with external threading and the stent graft system components are shown with internal threading, the threading may be reversed or any combination of threading may be used (e.g., each system may include a mix of internal/external). The first connection can be made by screwing the stent graft cover 36 to and about the delivery system outer cover 32. The second connection can be made by screwing the stent graft middle member 34 to and about the delivery system middle member 30. The terms first and second connection are merely identifiers and do not imply an order of connection. In one embodiment, the connections may be established at the same time (e.g., simultaneously). With threading, for example, the first and second attachments are detatchable. In other words, the delivery system can be removed from the stent graft system by unscrewing the first and second connection.

The stent graft system also includes a stent graft 50. The stent graft 50 can be self-expanding, in that it includes structures that are shaped or formed from a material that can be provided with a mechanical memory to return the structure from a compressed or constricted delivery configuration to an expanded deployed configuration. The stent graft includes two main components: a tubular graft 52, and one or more stents 54 for supporting and expanding the graft. The graft 52 may be formed from any suitable graft material, for example and not limited to, a low-porosity woven or knit polyester, DACRON material, expanded polytetrafluoroethylene, polyurethane, silicone, or other suitable materials. In another embodiment, the graft material could also be a natural material such as pericardium or another membranous tissue such as intestinal submucosa. The stent 54 is radially-compressible and expandable, is coupled to the graft material for supporting the graft material, and is operable to self-expand into apposition with the interior wall of a body vessel (not shown). Each stent 54 is constructed from a self-expanding or spring material, such as but not limited to Nitinol, stainless steel, a pseudo-elastic metal such as a nickel titanium alloy or nitinol, various polymers, or a so-called super alloy, which may have a base metal of nickel, cobalt, chromium, or other metal, or other suitable material. The stent 54 may be a sinusoidal patterned ring including a plurality of crowns or bends and a plurality of struts or straight segments with each crown being formed between a pair of opposing struts.

Referring to FIGS. 1 and 2, an operation of the catheter can be performed as follows. Prior to a surgical procedure, the stent graft middle member 34 can be attached to the delivery system middle member 30 via the second connection, and the stent graft cover 36 can be attached to the delivery system outer cover 32 via the first connection. A guide wire may be inserted into a vessel of the patient. The catheter 10 may be slid along the guidewire, whereupon the tapered tip 26 and middle members 30, 34 are fed along the guidewire to a desired location. Then, the surgical technician can withdraw or rotate the handle 16 of the catheter, thereby retracting the stent graft cover 36 toward the operator (while the middle member remains in place) and allowing the stent graft 50 to deploy and expand. Once the stent graft 50 is fully deployed and separated from the catheter, the technician can retract the entire catheter 10 from the body, followed by a removal of the guidewire.

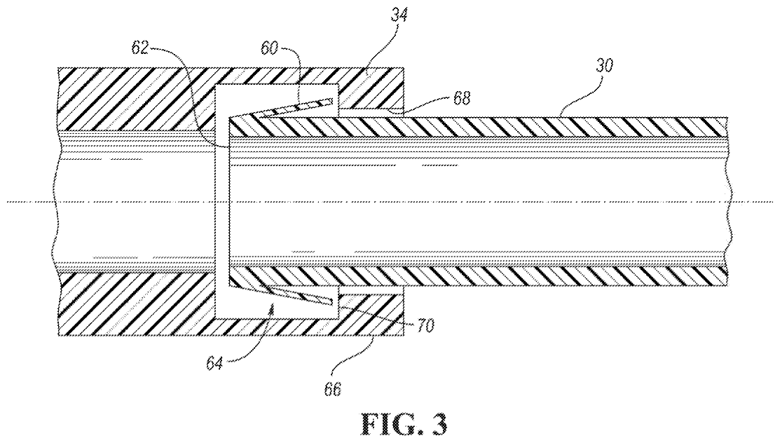

The first and second connections can be a threaded, screw connection as explained above. In another embodiment, the first and second connections are provided with a snap-fit connection. FIG. 3 shows one embodiment incorporating a snap-fit. In this embodiment, a connection between the delivery system middle member 30 and the stent graft middle member 34 is shown. However, it should be understood that a similar snap-fit connection can be made between the delivery system outer cover 32 and the stent graft cover 36. The delivery system middle member 30 includes one or more fingers or prongs 60 extending radially outward therefrom at a distal end 62 of the delivery system 12. The prongs 60 may be made of metal or plastic, and of a flexible nature such that the prongs are configured to bend inwardly when being inserted into the stent graft middle member 34.

The stent graft middle member 34 includes a pocket or cavity 64 at a proximal end 66 thereof. The cavity 64 may include a cut-out or pocket extending radially outward from the central opening of the stent graft middle member 34. During insertion of the delivery system middle member 30 into the stent graft middle member 34, the prongs 60 are forced to bend radially inwardly as the delivery system middle member 30 passes through the central opening 68 of the stent graft middle member 34. Then, as the prongs 60 pass axially beyond a ledge 70 formed in the stent graft middle member 34, they are allowed to “snap” and expand radially outwardly into the pocket 64. The delivery system middle member 30 is thereby locked in place, as an attempted forced removal of the delivery system middle member 30 from the stent graft middle member 34 is inhibited due to the prongs 60 contacting the ledge 70.

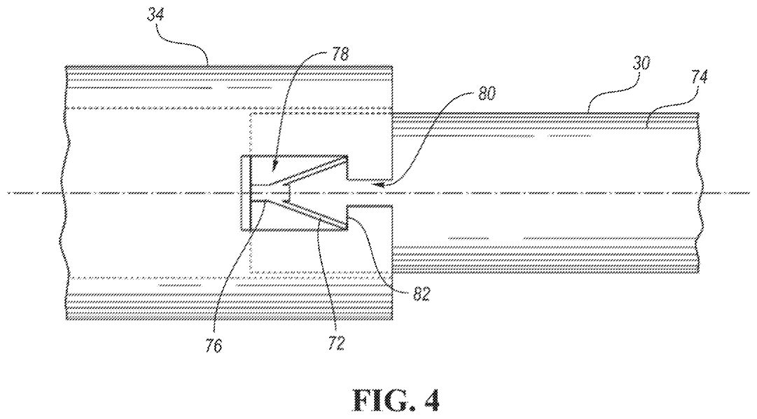

FIG. 4 shows another embodiment of a snap-fit connection. Once again, while FIG. 4 shows a connection between the delivery system middle member 30 and the stent graft middle member 34, it should be understood that a similar snap-fit connection can be made between the delivery system outer cover 32 and the stent graft cover 36. In this embodiment, the delivery system middle member 30 includes one or more fingers or prongs 72 at a distal end 62 thereof, made with similar properties as the prongs of FIG. 3. These prongs 72 can be molded with or attached to an outer surface 74 of the delivery system middle member 30. A pair of the prongs 72 can be connected at a joint 76 and extend axially along the outer surface 74 of the delivery system middle member 30. Rather than flexing or bending radially inwardly and outwardly (as in FIG. 3), the prongs 72 are configured to bend or flex in a direction generally tangential to the outer circumference of the delivery system middle member 30.

The stent graft middle member 34 includes a pocket 78 formed therein. This pocket may be a groove, slot, aperture, or other type of void of material extending through the stent graft middle member 34. The pocket includes a narrow inlet 80 at a proximal end 66 thereof. The inlet 80 is sized to allow the joint 76 to pass therethrough, along with the prongs 72 that flex toward one another during insertion. Then, as the prongs 72 pass axially beyond the inlet 80, the prongs 72 are allowed to expand outward from one another (as shown in FIG. 4), where they can contact a ledge 82 within the pocket 78. Removal of the delivery system middle member 30 from the stent graft middle member 34 is therefore inhibited as the prongs 72 contact the ledge 82.

This snap fit can be a removable connection. For example, since the prongs 72 are accessible from the exterior of the middle members 30, 34, a user can use a small tool to press or crimp the prongs 72 toward each other until they can fit through the inlet 80 of the pocket 78. Then, the delivery system middle member 30 can be removed from the stent graft middle member 34 in the axial direction, as the prongs 72 and joint 76 pass through the inlet 80. To access the removable connection of the middle members, the delivery system outer cover 32 and the stent graft cover 36 may be decoupled, first. For the snap fit shown in FIG. 3, one or more windows may be provided in the wall of middle member 34 to similarly allow access of a small tool to press the prong(s) 60 radially inward to allow the middle members to be released from each other (the same may apply to stent graft cover 36, if a snap fit is used). While several examples are described of ways to decouple snap fits, any suitable method may be used. Further, while the snap fit examples of FIGS. 3 and 4 show the stent graft system receiving the prongs (e.g., female component) and the delivery system having the prongs (e.g., male component), the configuration may be reversed.

The first and second connections have been described above as being either a threaded connection or a snap-fit connection. It should be understood that these connections are not necessarily exclusive of one another. For example, in a single catheter, the first connection can be a threaded connection, while the second connection can be a snap-fit connection, or vice versa. Moreover, a threaded connection can be incorporated into a snap-fit connection. For example, a small (e.g., less than full turn, such as quarter-turn) sized threaded connection can be incorporated into a snap-fit connection, thus requiring a, e.g., quarter turn of the middle members while inserting the flexible prongs of one middle member into the pocket of the other middle member.

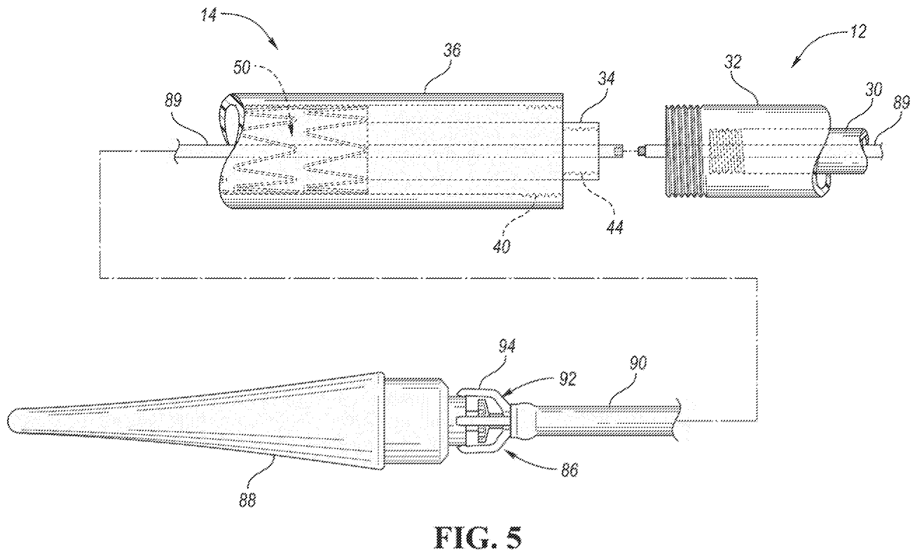

FIG. 5 illustrates a catheter being provided with a tip capture mechanism. Once again, the catheter is shown with a delivery system 12 that is separately connectable and assemblable to a stent graft system 14, with the delivery system outer cover 32 attachable to the stent graft cover 36 at the first connection, and the delivery system middle member 30 attachable to the stent graft middle member 34 at the second connection. The teachings of these two connections as explained above can be incorporated into the catheter of this embodiment.

The catheter includes a tapered tip 88 extending from a distal end of the catheter. The tapered tip 88 is the leading end of the catheter during insertion into the blood vessel, and includes a central opening for traveling over the guidewire. Adjacent to the tapered tip 88 is a tip capture mechanism 86, also referred to as a tip capture assembly, which includes an inner lumen or inner tube 89, and an outer lumen or outer tube 90. The inner tube 89 is fixed to the tapered tip 88 such that they move in unison. The outer tube 90 is slidable along the inner tube 89; the inner tube 89 is received within the outer tube 90 in a sliding manner. The outer tube 90 is connected to the handle 16 such that a button or release mechanism on the handle 16 can withdraw the outer tube 90 toward the technician while the inner tube 89 remains in place. The distal end of the outer tube 90 includes a tip capture spindle 92 which has fingers 94 or prongs. The tip capture spindle 92 is configured to hold a stent, ring, loop, or other such structure of proximal end of the stent graft 50. This allows the stent graft 50 to be deployed while its proximal end is held in a constricted manner during deployment. Once the stent graft 50 is at least partially deployed, the outer tube 90 can be slid relative to the inner tube 89 via the handle 16, releasing the fingers 94 or prongs of the tip capture spindle 92 from the stent, ring, loop, or the like of the stent graft 50. This releases the stent graft 50 from the catheter, and the entire catheter can then be removed from the patient. While one example of a tip capture mechanism has been described, any tip capture mechanism may be compatible with the present disclosure. For example, the tip capture mechanism may be configured such that the inner tube extends axially forward to move the tip forward relative to the outer tube and spindle, thereby releasing the stent graft. Other tip capture mechanisms may include a single tube, three or more tubes, or other systems.

In at least one embodiment, the outer tube 90 has a connection (e.g., threaded, snap-fit, etc.) similar to the first and second connections described above. This creates a stent graft portion of the outer tube 90 that is separately connected to a delivery system portion of the outer tube 90. In other embodiments, the outer tube 90 of the tip capture assembly 86 can extend entirely through the catheter as a singularly-formed tube. Likewise, the inner tube 89 can be either a single unitary tube, or, as illustrated in FIG. 5, can have two portions separately connected at a similar threaded or snap-fit connection (only inner tube 89 is shown with a connection, to simplify the illustration, however, an additional connection may be present for outer tube 90.

The connection between the delivery system 12 and the stent graft system 14 (e.g., the first and second connections) can be located adjacently proximal to the stent graft 50 (e.g., near the back/proximal end of the stent graft, as loaded). Alternatively, the connection can be located at a point along the catheter 10 that balances the length of each packaged portion to optimize the connection location. In other words, the connection point can be located at a midway point between the proximal end of the handle 16 and the distal end of the tapered tip 88. This can create an even packaging space, whereupon the delivery system 12 and stent graft system 14 each take up a relatively equal length in packaging. In yet another embodiment, the connection can be located adjacent to the distal end of the handle 16. This would allow larger connections (e.g., larger threading or snap-fit pieces) since they would not be inserted into the patient during a surgical procedure. In other words, since the first and second connections are located closer to the handle, they will not be inserted into the patient's body during surgery but will instead remain external to the body. This removes any size constraints of the connections, allowing the connections to be larger and more robust since they need not fit into inherent size constraints of blood vessels. This also allows the surgical technician (or other operator) to easily disassemble one stent graft system 14 from the delivery system 12, and reassemble another stent graft system. This may be helpful in a dual-stent surgical procedure, for example.

The ability to separately deliver and subsequently assemble the delivery system 12 with the stent graft system 14 provides a benefit of a reduction of necessary SKUs. Multiple catheter variations can be made available, allowing the user to select the optimal delivery system and stent graft system separately. Variations in catheter length or flexibility/stiffness can be made available to connect with different stent graft systems in a plethora of combinations. Attempting to accomplish this with a single fully-assembled system would require a corresponding number of SKUs. For example, a set of nine different stent graft diameters and four different overall catheter lengths would require 36 separate SKUs if the catheter were a single fully-assembled system, but would only require 13 SKUs if the delivery system can be provided separately from the stent graft system.

The ability to separately deliver and subsequently assemble the delivery system 12 with the stent graft system 14 also enables storage of the stent graft system 14 in alternative materials, such as liquid. For example, while the delivery system 12 can be shipped and/or stored in a dry container, the stent graft system 14 can be shipped and/or stored in a liquid. Storage of the stent graft system 14 in liquid, and keeping air from being trapped within the stent graft system, can reduce or eliminate air escaping from the stent graft system 14 and into the patient's body during deployment of the stent graft. This may also reduce or eliminate the need to flush the stent graft system 14 prior to the procedure. Additionally, this may reduce the chances of infection, maintain sterility, preserve the device, and reduce friction for ease of deployment of the stent graft. Examples of liquid that the stent graft system 14 can be stored in include, but are not limited to, saline, glutaraldehyde, or various disinfectants, preservatives, antibiotics, medications, lubricants, etc. that are not harmful if released into the blood vessel upon deployment.

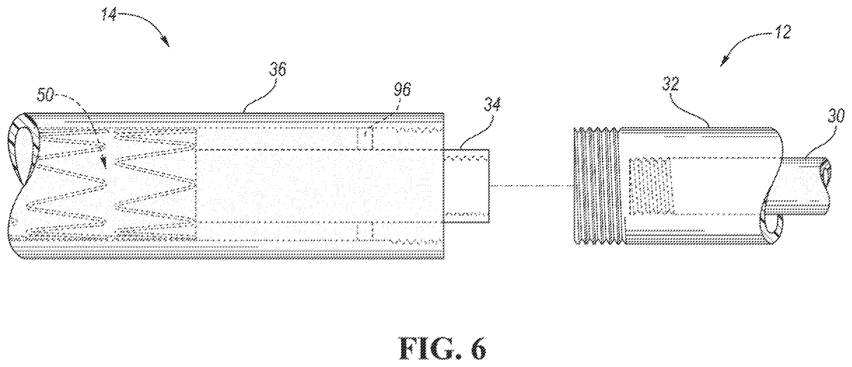

If the stent graft system 14 is to be stored in a liquid, a seal may be implemented. FIG. 6 illustrates one example of a seal located in the stent graft delivery system 14. The seal 96 can be an annular seal located radially between the stent graft middle member 34 and the stent graft cover 36. The seal 96 can be of sufficient flexibility to allow the stent graft cover 36 to be slid in the axial direction over the seal 96 during deployment of the stent graft. The seal 96 can be fixed to the stent graft middle member 34 to allow the stent graft cover 36 to slide in such a manner. Alternatively, the seal 96 can be fixed to the stent graft cover 36, and can be slid across the stent graft middle member 34 during deployment of the stent graft 50. The seal 96 can be made of rubber or other synthetic, flexible materials.

While exemplary embodiments are described above, it is not intended that these embodiments describe all possible forms encompassed by the claims. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

1: An endovascular catheter comprising:
a stent graft system having:
a stent graft configured to expand radially outwardly,
a stent graft cover surrounding at least a portion of the stent graft and configured to maintain the stent graft in a constricted configuration, and to slide relative to the stent graft to enable the stent graft to expand radially outward, and
a stent graft middle member located radially inward of the stent graft cover, the stent graft middle member being hollow; and
a delivery system configured to couple to the stent graft system and having:
a delivery system outer cover configured to assemble to the stent graft cover via a first connection, and
a delivery system middle member configured to assemble to stent graft middle member via a second connection.

2: The endovascular catheter of claim 1, wherein the first connection is a threaded connection in which the delivery system outer cover has threads engageable with corresponding threads of the stent graft cover.

3: The endovascular catheter of claim 1, wherein the second connection is a threaded connection in which the delivery system middle member has threads engageable with corresponding threads of the stent graft middle member.

4: The endovascular catheter of claim 1, wherein at least one of the first connection and second connection is a snap-fit connection.

5: The endovascular catheter of claim 4, wherein the first connection is a snap-fit connection in which the delivery system outer cover includes one or more flexible fingers, and the stent graft cover includes a cavity, wherein the flexible fingers are configured to flex radially inwardly during insertion of the delivery system outer cover into the stent graft cover and flex radially outwardly into the cavity to provide the snap-fit connection.

6: The endovascular catheter of claim 4, wherein the second connection is a snap-fit connection in which the delivery system middle member includes one or more flexible fingers, and the stent graft middle member includes a cavity, wherein the flexible fingers are configured to flex radially inwardly during insertion of the delivery system middle member into the stent graft middle member and flex radially outwardly into the cavity to provide the snap-fit connection.

7: The endovascular catheter of claim 1, wherein the stent graft middle member is configured to receive therein an inner tube disposed within an outer tube, wherein the outer tube coupled to a tip capture mechanism releasable from the stent graft as the outer tube slides relative to the inner tube, and wherein the inner and outer tubes extend through the first and second connections.

8: The endovascular catheter of claim 7, wherein the inner tube includes a first inner tube part releasably connected to a second inner tube part via an inner tube releasable connection, and wherein the outer tube includes a first outer tube part releasably connected to a second outer tube part via an outer tube releasable connection.

9: The endovascular catheter of claim 1, further comprising an annular seal disposed radially between the stent graft cover and the delivery system middle member.

10: An endovascular catheter assembly comprising:
a stent graft portion having a proximal end, a distal end, an expandable stent graft, and a stent graft cover configured to slide over the stent graft to enable the stent graft to expand radially outward; and
a delivery portion having a proximal end, a distal end, a handle at the proximal end of the delivery portion, and an outer sleeve extending from the handle toward the distal end of the delivery portion, wherein the outer sleeve is configured to assemble to the stent graft cover.

11: The endovascular catheter assembly of claim 10, wherein the outer sleeve is configured to assemble to the stent graft cover via a threaded connection.

12: The endovascular catheter assembly of claim 10, wherein the outer sleeve is configured to assemble to the stent graft cover via a snap-fit connection.

13: The endovascular catheter assembly of claim 10, wherein the stent graft portion includes a stent graft middle member, and the delivery portion includes a delivery middle member, wherein the stent graft middle member and the delivery middle member are hollow.

14: The endovascular catheter assembly of claim 13, wherein the outer sleeve is configured to assemble to the stent graft cover via a first connection, and the stent graft middle member is configured to assemble to the delivery middle member via a second connection.

15: The endovascular catheter assembly of claim 14, wherein at least one of the first and second connections is a threaded connection.

16: The endovascular catheter assembly of claim 15, wherein at least one of the first and second connections is a snap-fit connection including flexible fingers that are configured to flex inwardly and outwardly.

17: The endovascular catheter assembly of claim 10, wherein the outer sleeve is configured to assemble to the stent graft cover at a location closer to the handle than the proximal end of the stent graft portion.

18: The endovascular catheter assembly of claim 10, wherein the stent graft cover and the outer sleeve have different stiffnesses.

19: A method of deploying a plurality of stent grafts in one or more vessels of a body, the method comprising:
assembling a first stent graft system to a delivery system in which a first stent graft cover is removably coupled to a delivery system outer cover;
advancing the first stent graft system to a first deployment site within the body;
retracting the first stent graft cover to enable a first stent graft to deploy;
withdrawing at least a portion of the first stent graft system and the delivery system away from the body;
removing the first stent graft cover from the delivery system outer cover;
assembling a second stent graft system to the delivery system in which a second stent graft cover is removably coupled to the delivery system outer cover;
advancing the second stent graft system to a second deployment site within the body; and
retracting the second stent graft cover to enable a second stent graft to deploy.

20: The method of claim 19, wherein at least one of the steps of assembling includes threadingly connecting.
